(19) United States
(12) Patent Application Publication
(10) Pub. No.: US 2023/0037424 A1
(43) Pub. Date: Feb. 9, 2023

(54) A SYSTEM AND METHOD FOR CLASSIFYING IMAGES OF RETINA OF EYES OF SUBJECTS

(71) Applicant: Mona.health, Bertem (BE)

(72) Inventor: Bart ELEN, Mol (BE)

(21) Appl. No.: 17/909,073

(22) PCT Filed: Dec. 15, 2020
(86) PCT No.: PCT/EP2020/086107

§ 371 Date: Sep. 2, 2022

(30) Foreign Application Priority Data
Mar. 3, 2020 (EP) 20160697.7

(51) Int. Cl. G06T 7/00; A61B 3/00; G06T 7/11; G06V 40/18

(52) U.S. Cl. CPC G06T 7/0012; A61B 3/0025; G06T 7/11; G06V 40/18; G06T 2207/10024; G06T 2207/20081; G06T 2207/30041; G06T 2207/30101

(57) ABSTRACT

The invention relates to a computing system and a computer-implemented method for classifying images of retina of eyes of subjects. A captured image of a retina is processed to obtain a plurality of different segmented images each having different selected portions of the captured image using different selection rules. The multiple segmented images are provided to respective dedicated machine learning models to output an image classification based on the respective segmented images provided as input. An ensemble classification is determined based on the multiple classifications obtained by means of the multiple trained machine learning models.

### FIELD OF THE INVENTION

The invention relates to a system and a computer-implemented method for classify images of retina of eyes of subjects. Further, the invention relates to an imaging apparatus and a use.

### BACKGROUND TO THE INVENTION

The retina is the innermost, light-sensitive layer of tissue of the eye of humans and some animals. The optics of the eye create a focused two-dimensional image of the visual world on the retina, which translates that image into electrical neural impulses to the brain to create visual perception, the retina serving a function analogous to that of the film or image sensor in a camera.

The retina enables visual imaging of the microvasculature in a non-intrusive way. Retinal diseases or conditions tend to go unnoticed for a long time, as it initially may not cause noticeable symptoms for a patient. It is often only when the eye sight is affected that the ophthalmologist detects the medical condition during an eye exam. This can put significant pressure on the health care system, and calls for a more preventive and more efficient approach.

It is known that dimensions of retinal vessels can change in certain conditions or ocular diseases, such as diabetes, coronary heart disease, stroke and Alzheimer's disease. For instance, diabetic retinopathy and glaucoma are lead causes of preventable, but incurable blindness. Glaucoma can be spotted by examining whether the optic disc is cupping, while diabetic retinopathy can be linked with the presence of hard exudates on the retina, among other symptoms. At the same time, there can be small aberrations observable in the retinal vasculature. For instance, in the case of diabetic retinopathy, changes have been recorded in the retinal blood flow and arteriolar tortuosity. Furthermore, evaluation of retinal vasculature has been proposed as an easy and complementary step in the screening process for cardiovascular disease. For example, the first sign of diabetic retinopathy can be a presence of microaneurysms.

Large scale retinal screenings could be used as part of a regular health checkup for early condition/disease identification and treatment. The analysis of retina images can be a complex and labor-intensive manual task requiring a highly-skilled clinician. Automated analysis of the images can speed up the screening process, and remove the human error component. Deep learning and convolutional neural networks (CNNs) have been successfully applied for identifying and extracting the microvasculature from fundus images in an automated setting.

There is a need for improving the interpretability and/or explainability of the results obtained by the deep learning models. Furthermore, there is a desire to increase the robustness of the systems and methods used, and to better quantify the confidence level of the prediction of the model.

### SUMMARY OF THE INVENTION

It is an object of the invention to provide for a method and a system that obviates at least one of the above mentioned drawbacks.

Additionally or alternatively, it is an object of the invention to improve systems and methods for retina analysis.

Additionally or alternatively, it is an object of the invention to improve the interpretability and/or explainability in systems and methods for retina analysis.

Additionally or alternatively, it is an object of the invention to improve the robustness of the systems and methods for retina analysis.

Additionally or alternatively, it is an object of the invention to improve the prediction confidence level quantification of the systems and methods for retina analysis.

Additionally or alternatively, it is an object of the invention to provide for systems and methods which can more accurately detect retinal disease, preferably also in an early stage.

Thereto, the invention provides for a computing system configured to classify images of retina of eyes of subjects, the system including one or more hardware computer processors, and one or more storage devices configured to store software instructions configured for execution by the one or more hardware computer processors in order to cause the system to: receiving an initial image of a retina captured by means of an imaging unit; processing the initial image of the retina in order to obtain at least a first segmented image and a second segmented image different from the first segmented image, wherein the first segmented image only includes a first selected portion of the captured image of the retina by employing a first selection rule, and wherein the second segmented image only includes a second selected portion of the captured image of the retina by employing a second selection rule, the first and second selection rules being different with respect to each other; providing at least a first trained machine learning model and a second trained machine learning model each configured to output an image classification based on an input image, the first and second machine learning models being different with respect to each other, and wherein the first machine learning model is trained using segmented images with only selected portions obtained by employing the first selection rule, and wherein the second machine learning model is trained using segmented images with only selected portions obtained by employing the second selection rule; providing the first segmented image with the first selected portion to the first machine learning model as input image in order to obtain a first classification, and providing the second segmented image with the second selection portion as input image to the second machine learning model as input image in order to obtain a second classification; and determining an ensemble classification based on at least the first classification and the second classification.

The retina image being captured (cf. fundus picture) can be processed employing a plurality of different segmentation selection rules in order to obtain a plurality of segmented images. The first segmented image can be provided as input to a respective first machine learning model which is trained using training images which were processed similarly using the first selection rule. Similarly, the other segmented images (e.g. second segmented image) can be provided as input to a respective other machine learning models (different with respect to at least the first machine learning model) that are each trained using training images which were processed using the other respective selection rules. The at least two different machine learning models can be for example convolutional neural networks (cf. deep learning model) suitably trained to detect one or more conditions of the subject (e.g. disease, age, etc.). A collection or set of differently trained networks can be employed, each based on different selection rules. Each of the plurality of networks may be trained to predict the same condition (e.g. same possible classification outputs, for instance related to glaucoma). In this way, each trained machine learning model may be trained to particularly use or focus on a different portion of information which is present in the captured image of the retina. For example, the trained machine learning model may only receive segmented images obtained by applying a selection rule for selecting only regions corresponding to the vessel structures in the captured image of the retina. Additionally or alternatively, for example, a trained machine learning model may only receive segmented images obtained by applying a selection rule for selecting only regions corresponding to the optic disc in the captured image of the retina. Additionally or alternatively, for example, a trained machine learning model may only receive segmented images obtained by applying a selection rule for selecting all regions except the region covering the optic disc in the captured image of the retina. Additionally or alternatively, it is also possible to use other trained machine learning models in parallel which are configured to receive unsegmented images as input (for example the captured image without excluded regions; black-and-white image of the captured image; etc.).

Advantageously, a separate machine learning model can be trained to perform a classification and/or predication based on selected “portions/regions” of the image using preset/predefined selection rules. The selection rules may take into account features of the retina image (e.g. optic disc, vessel structure, ring surrounding the optic disc, etc, and/or a combination of such features).

If the first trained machine learning model provides, based on a first segmented image, a first classification with a high confidence level, it can be derived which changes happened in the second segmented image which can be related to the first classification provided by the first trained machine learning model, and/or vice versa. For example, a first machine learning model may be trained to detect glaucoma from a segmented image which only includes regions covering the vessel structure in the captured retina image. This means that it is not only possible to detect whether the subject has glaucoma, but also able to say which change has happened which can be linked to glaucoma. For instance, if the first trained machine learning model is very confident that the subject has glaucoma, this would indicate that changes to the vessel structure happened related to glaucoma. This may also provide a better way to train the second machine learning model (cf. other machine learning models used in parallel with the first machine learning model). Therefore, in addition to increased accuracy, it is also possible to vastly enhance the explainability and interpretability, due to changes to optic disc, due to change to the vessels, etc. The overall predications and/or classifications can be made more trustworthy in an advantageous way.

The selection rules may be employed manually or automatically. An automatic application of the selection rules may involve machine learning models for example (e.g. for selection blood vessels). However, pre-programmed rule-based methods may also be used (e.g. for selecting the optic disc in the retina image). Application of the selection rules may involve cropping or cutting actions for retaining only desired regions in the captured retina image. It is also possible to cover the images or neutralizing data within certain regions in the captured image of the retina. It is envisaged that this can be done in various ways.

Optionally, the first and second selection rules are configured such that the first and second selected portions of the captured image have no overlap.

In this way, the different segmented images may cover different regions within the captured image of the retina. It can be effectively prevented that overlapping hotspots are used to come to the same classification. By preventing overlap, it can be better guaranteed that different information present in different portions of the captured retina image are used for classification.

Optionally, the second selection rule is configured to provide a fully inverted selection with respect to the first selection rule. In this way, overlap can be prevented in an easy way.

Optionally, the second selection rule is configured to provide at least partially inverted selection with respect to the first selection rule.

In this way, the second selection rule can be coupled with the first selection rule, providing more consistent results. It can also better be assessed what the overlapping regions are by applying the first and second selection rules. Partial inversion may be required when more than three segmented images are generated with more than three respective selection rules. In some examples, overlap is prevented taking into account all the segmented images generated by all the different selection rules.

Optionally, the first selection rule is based on identification of an optic nerve head of the eye, and wherein the second selection rule is based on an identification of blood vessels of the eye.

Optionally, the first selection rule is configured to selectively exclude a region covering an identified optic nerve head of the eye in the captured initial image, wherein the second selection rule is configured to selectively include a region covering the identified optic nerve head of the eye in the captured initial image.

By excluding the optic nerve head or optic disc region, it can be better guaranteed that also other information not covered by the optic disc region in the captured retina image is used. Typically, when a machine learning model is used to receive the unsegmented image of the retina as input, mainly information in the optic disc region in the retina image is used by the machine learning model for generating the output classification. According to the invention, it is also possible to effectively use other portions/parts of the captured retina image for classification.

Optionally, the first selection rule is configured to selectively exclude a region covering identified blood vessels of the eye in the captured initial image, wherein the second selection rule is configured to selectively include a region covering the identified blood vessels of the eye in the captured initial image.

In some cases, the interpretability can be increased by separately taking into account regions covering blood vessels in the retina image. Advantageously, the machine learning model is particularly trained using the same selection rules, resulting in more accurate predications while allowing to more effectively ‘looking’ at different regions of the retina image (instead of mainly the optic disc).

Optionally, the initial image of the retina is processed in order to obtain one or more further segmented images, wherein each of the one or more further segmented images only includes a further selected portion of the captured image of the retina by employing a further selection rule, the further selection rule being different with respect to other selection rules being employed; wherein one or more further trained machine learning models are provided configured to output an image classification based on an input image, each of the one or more further trained machine learning models being different with respect to other trained machine learning models being provided, wherein each of the one or more further machine learning models is trained using further segmented images with only further selected portions obtained by employing the further selection rule; wherein the one or more further segmented images are provided to the respective further machine learning models as input image in order to obtain one or more respective further classifications; wherein the ensemble classification is further based on the one or more further classifications.

Optionally, at least two further segmented images are generated each provided as input to at least two respective machine learning models.

Optionally, at least four different trained machine learning models are employed each configured to receive a respective different segmented image obtained by employing different respective selection rules based on one or more eye features in the captured image.

In some examples at least four segmented images are generated employing different selection rules. More segmented images with different selection rules may also be employed (for instance six, eight, ten). In this way, the interpretability and/or explainability can be further enhanced.

Optionally, at least two of the following selection rules are employed: exclusion of region covering an identified optic disc in the captured initial image; exclusion of region covering identified blood vessels in the captured initial image; inclusion of region only covering an identified optic disc in the captured initial image; and inclusion of region only covering identified blood vessels in the captured initial image.

Optionally, segmentation in segmented images is performed by removing image data within at least one segment area of the captured image of the eye, the at least one segment area covering parts of the eye to be excluded.

The at least one segment area may have various shapes and forms, e.g. circular, ellipsoid, rectangular, square, polygonal. Other shapes are also possible, for instance custom shapes covering relevant landmarks/features of the retina.

Optionally, removing of image data is performed by applying a patch over the at least one segment area.

Optionally, the image data within the segment area is removed by setting at least one of a normal distribution, a random distribution, or a uniform distribution of pixel values within the at least one segment area of the captured image.

As a result, when the segmented image is provided to one or more trained neural networks/models, the one or more segmented areas therein may not contain relevant information for classification. Hence, the trained machine learning model can focus on other parts of the segmented image.

Optionally, the unsegmented captured image is further provided as input to the one or more trained machine learning models for classifying the eye.

Optionally, the classification is usable to infer or further analyze a condition of the subjects.

According to an aspect, the invention relates to a computing system configured to classify images of retina of eyes of subjects, the system including one or more hardware computer processors, and one or more storage devices configured to store software instructions configured for execution by the one or more hardware computer processors in order to cause the system to: receiving an initial image of a retina captured by means of an imaging unit; processing the initial image of the retina in order to obtain at least a first processed image and a second processed image different from the first processed image, wherein the first processed image only includes a first selected portion of information of the captured image of the retina by employing a first processing rule, and wherein the second processing image only includes a second selected portion of information of the captured image of the retina by employing a second processing rule, the first and second processing rules being different with respect to each other; providing at least a first trained machine learning model and a second trained machine learning model each configured to output an image classification based on an input image, the first and second machine learning models being different with respect to each other, and wherein the first machine learning model is trained using processed images with only selected portions of information obtained by employing the first processing rule, and wherein the second machine learning model is trained using processed images with only selected portions of information obtained by employing the second processing rule; providing the first processed image with the first selected portion of information to the first machine learning model as input image in order to obtain a first classification, and providing the second processed image with the second selection portion of information as input image to the second machine learning model as input image in order to obtain a second classification; and determining a combined/ensemble classification based on at least the first classification and the second classification.

The first selected portion of information may for instance be obtained by using a color filter, spatially deleting/neutralizing information in selected regions/areas (e.g. selection rule), selectively deleting/neutralizing information in images obtained by performing any kind of predetermined data processing employed on the captured image (e.g. frequency analysis processing, spectral analysis processing, proper orthogonal decomposition analysis processing, rule-based image processing, image filtering, image processing by means of machine learning, singular value decomposition processing, etc.). Many types of (data/image) processing techniques known in the art may be employed for this purpose.

According to an aspect, the invention provides for a computer-implemented method of classifying images of retina of eyes of subjects, the method comprising operating one or more hardware processors to: receiving an initial image of a retina captured by means of an imaging unit; processing the initial image of the retina in order to obtain at least a first segmented image and a second segmented image different from the first segmented image, wherein the first segmented image only includes a first selected portion of the captured image of the retina by employing a first selection rule, and wherein the second segmented image only includes a second selected portion of the captured image of the retina by employing a second selection rule, the first and second selection rules being different with respect to each other; providing at least a first trained machine learning model and a second trained machine learning model each configured to output an image classification based on an input image, the first and second machine learning models being different with respect to each other, and wherein the first machine learning model is trained using segmented images with only selected portions obtained by employing the first selection rule, and wherein the second machine learning model is trained using segmented images with only selected portions obtained by employing the second selection rule; providing the first segmented image with the first selected portion to the first machine learning model as input image in order to obtain a first classification, and providing the second segmented image with the second selection portion as input image to the second machine learning model as input image in order to obtain a second classification; and determining an ensemble classification based on at least the first classification and the second classification.

Advantageously, the invention employs multiple machine learning models, each trained to perform the predication in parallel. The multiple machine learning models are each trained by using training images on which respective selection rules are applied (different selection rule for each trained machine learning model). The results of the multiple machine learning models (cf. classifiers) can be combined to obtain the ensemble classification, effectively taking into account different portions of the initial captured retina image using the selection rules.

The multiple machine learning models can be arranged in parallel. Advantageously, the model robustness, interpretability, explainability and confidence quantification can be significantly enhanced by using the plurality of trained machine learning models in parallel, at least a subset of the plurality of machine learning models using different input data (cf. different segmented images obtained by application of different selection rules). According to the invention, a set of different machine learning models can be trained taking into account the different segmentation selection rules. It can be better guaranteed that different parts of the information of the captured retina image is provided as input to the respective trained machine learning models.

At least a subset of the total number of machine learning models employed may be trained on different parts of information of the image of retina (predefined using the segmentation selection rules.

According to an aspect, the invention provides for an imaging apparatus comprising the system according to the invention, and an imaging unit for capturing images of the retina. Furthermore, according to an aspect, the invention provides for a use of the imaging apparatus for classifying images of retina of eyes of subjects.

According to an aspect, the invention provides for a computing system and a computer-implemented method for classifying images of retina of eyes of subjects. The system including one or more hardware computer processors, and one or more storage devices configured to store software instructions configured for execution by the one or more hardware computer processors in order to cause the system to: receive an image of a retina captured by means of an imaging unit, the captured image including an optic nerve head of the eye; provide the captured image as input to one or more trained machine learning models configured to classify the eye, wherein the one or more trained machine learning models provide a first classification of the eye as output; segment the captured image of the retina in order to obtain a segmented image which is segmented to exclude the optic nerve head of the eye; provide the segmented image as input to the one or more trained machine learning models, wherein the one or more trained machine learning models provide a second classification of the eye as output; and determine an ensemble classification based on the first classification and the second classification.

A first classification is performed with the unsegmented image including the optic nerve head (as the trained neural network tends to primarily use information in the image present in the area covered by the optic nerve head for the classification), and a second classification is performed with the segmented image excluding the optic nerve head (forcing the trained neural network to use data elsewhere in the image). The final/ensemble classification is based on the first and second classifications.

The trained neural network machine learning models have a tendency to primarily ‘look’ at particular regions of the retinal fundus image (e.g. mainly to the optical disc) for determining its output. The solution provided by masking/neutralizing data covered by hotspots typically used by the trained machine learning model (e.g. by segmenting optical disc away), in order to force the trained machine learning model to use other data within the retinal fundus image provides significant advantages with regard to interpretability, explainability and/or robustness. Advantageously, additionally or alternatively, the trustworthiness can be enhanced, as the retina image includes redundant information usable for detecting a certain classification (e.g. a condition/disease of the eye).

According to an aspect, the invention provides for a computing system configured to classify images of retina of eyes of subjects, the system including one or more hardware computer processors, and one or more storage devices configured to store software instructions configured for execution by the one or more hardware computer processors in order to cause the system to: receiving an image of a retina captured by means of an imaging unit; segmenting the captured image of the retina in order to obtain at least one segmented image; and providing the at least one segmented image as input to one or more trained machine learning models configured to classify the eye.

The system can employ one or more deep learning models for analyzing retinal images, wherein information in only parts of the initial image of the retina (e.g. excluding some segments) are provided as input to the one or more deep learning models. The deep learning models can be trained to automatically identify disease patterns or medical conditions in the segments of the retinal images. In this way, a classification may be performed by excluding one or more selected regions of the retina image (cf. only parts of retina image excluding some segment areas are provided to the trained neural network).

The at least one segment can correspond to selected portions of the complete image of the retina. Segmentation can be performed in different ways. For example, segmentation may be performed based on identified landmarks/features in the retina image, such as for instance at least one of: an optic nerve head, blood vessels (with or without differentiation between arteries and veins), subgroup of blood vessels (e.g. only the arteries, or only the veins, or only the vessels from one quadrant of the retina, and/or in a certain zone around the optic disc), eye nerves, regions of the retina, etc. Various segmentation algorithms can be employed. In some examples, segmentation is performed by means of image processing algorithms. Additionally or alternatively, one or more machine learning algorithms may be used for effective segmentation of the retina image (e.g. automatic detection of blood vessels).

Advantageously, according to the invention, it can be better determined which landmarks/features (e.g. optic disc, vessel structure, etc.) of the retina image has resulted to a positive detection of the disease/condition by using the one or more trained machine learning models provided with segmented images (with neutralized data within some segments in the (optionally preprocessed) captured image). Also, our approach allows to determine the relative importance of changes to different landmarks/features. The disease (e.g. glaucoma) will cause changes at different locations in the retina (optic disc, retinal nerve fiber layer, vessel structure, . . . ). Patients might, with the same degree of change in the optic disc, have a different change in retinal nerve fiber layer or vessel structure.

The claimed system can also provide for better explainability, as it can be better identified where in the retina image changes have occurred. Moreover, the model can provide for a higher interpretability. It can be better determined how the model is combining information from distinct retinal regions. For example, the model trust worthiness can be enhanced by realizing the retinal analysis based on distinct, but redundant retinal features.

Optionally, a first segmented image of the at least one segmented image is segmented to exclude an optic nerve head of the eye in the captured image.

In many cases, trained neural networks provided with a complete image of the retina for classification of the eye, mainly use information in the optic nerve head in the retina image. In this way, other portions of the retina image may only be minimally used. This can be prevented, by effectively segmenting the retina image such as to exclude the optic nerve head. The segmented image then includes the remaining portions of the retina. The trained neural network is thus forced to use the remaining portions of the retina image (excluding portions corresponding to the optic nerve head) for classification.

Optionally, a second segmented image of the at least one segmented image is segmented to only include an optic nerve head of the eye in the captured image.

In this way, the trained neural network can be forced to perform the classification by only taking into account portions of the retina image representing the optic nerve head, excluding other portions. In some examples, the resulting classification can be compared to one or more classifications obtained by using other segmented images of the retina (for instance excluding regions of the retina image representing the optic nerve head). Accordingly, it can be better pinpointed which portions/regions of the retina image have resulted to a certain classification of the retina image.

Optionally, a third segmented image of the at least one segmented image is segmented to only include blood vessels of the eye in the captured image.

By segmenting the retina image to exclusively include portions of the captured image corresponding to blood vessels of the retina, it can be better ensured that the trained neural network takes into account the blood vessels for the classification of the retina of the eye.

Optionally, only a subset of identified blood vessels of the eye in the captured image is used.

The arteries transport blood, rich in oxygen, towards body parts, while veins transport blood, low in oxygen, back to the heart. Arteries can be distinguished from veins in the retina due to difference in color (veins may be darker while arteries may be brighter). Also, they may tend to alternate in the retina. It can be valuable to make a distinction between arteries and veins when studying changes to the retinal vessel structure. For example, narrowing of retinal arteries and dilation of retinal veins can be known signs of increased cardiovascular risk.

The thickness of the retinal vessels can depend on the distance from the optical disc. The further away from the optical disc, the thinner the vessels may become. An arteriole-to-venule ratio can be defined as a difference in thickness between arterioles and venules. This ratio can change with the distance to the optical disc.

Optionally, a fourth segmented image of the at least one segmented image is segmented to exclude blood vessels of the eye in the captured image.

Optionally, at least two of the first, second, third and fourths segmented images are separately provided as input to the one or more trained machine learning models for classifying the eye, each output being linked to a subcategory.

By performing separate classifications using different segmented images focused on different parts of retina image, a more accurate prediction of a classification may be obtained. Additionally or alternatively, it is possible to better identify which landmarks/features of the eye (e.g. blood vessels, optic nerve head, particular regions of the eye, etc.) have resulted in a certain classification (e.g. glaucoma).

A trained machine learning model may tend to look at certain parts/regions of the retina image (heat spots). For instance, the trained machine learning model may mainly take into account the optic disc (cf. optic nerve head) of the retina image for detecting a disease or condition. By performing different classifications based on a plurality of segmented images of the retina, it can be avoided that the machine learning model has a hard focus on only certain parts or regions of the image of the retina (the rest of the retina can also be taken into account). By performing segmentation, parts of the image of the retina can be “discarded” prior to feeding it to a trained neural network. Information in a part/region of the image can be neutralized (e.g. replaced by a uniform color, random pixel values, etc.).

Optionally, the unsegmented captured image is further provided as input to the one or more trained machine learning models for classifying the eye.

Optionally, segmented classification outputs based on different segmented images provided as input to the one or more trained machine learning models are provided to an ensemble model for generating a general classification output.

For instance, this optic nerve may be cut out (segment), and then the trained machine learning model can still give a good prediction (with a good accuracy, but taking parts of the image of the retina into account different than the optic nerve). In this way, it can be forced that the employed trained neural network will look at a zone/region of the retina image that lies outside the optic disc (nerve). This may be medically related to a highly relevant observation. By performing segmentation (e.g. discarding information in zone/region of the optic disc), some part of the information in the retina image may be lost, resulting in a reduction of the performance of the neural network. However, by performing the analysis separately for two or more segmented images, and/or separately for at least one segmented image and at least one unsegmented image, a more accurate classification can be obtained. Furthermore, directly a better indication can be provided about which parts of the retina image contributed to detection of the disease or condition (e.g. optical disc, blood vessel structure, etc.).

Optionally, the captured image and/or at least one segmented image is preprocessed prior to providing it as input to the one or more machine learning models.

The preprocessing may involve for instance image preprocessing for enhancing the image to be provided to the one or more machine learning models. Various preprocessing steps can be carried out, such as for example sharpening, improving the contrast, applying image filters, perform noise reduction, etc.

Optionally, one or more features are extracted from the at least one segmented image, wherein the one or more extracted features are provided as input to one or more machine learning models.

Optionally, the image of the retina is a hyperspectral image. For instance, a hyperspectral camera can be used. The image of the retina can have more information.

Optionally, the imaging unit is integrated in the system.

Optionally, the classification is usable to infer or further analyze a condition of the subjects.

Exemplary eye diseases which can be detected from retinal images are diabetic retinopathy, glaucoma, macular degeneration. However, other diseases and disease risks can also be identified from retina images. Examples are systemic diseases (e.g. diabetes), neurological diseases (e.g. Alzheimer's disease), and cardiovascular diseases (e.g. stroke).

The system can more accurately determine these indications by employing segmented images of the retina image, excluding one or more portions and including one or more selected portions corresponding to some features and/or areas of the retina (e.g. optical disc head, vessel structure, subgroup of vessels, etc.). Changes to the vessel structure may be linked to diabetic retinopathy. Further, changes to structure (e.g. shape) of the optic disc and/or changes to within the optic disc (e.g. cupping) can be linked to glaucoma. Furthermore, changes at the retinal nerve fiber layer may be linked to glaucoma. Changes in an area around the macula may be linked to diabetic macular edema and/or age-related macular degeneration.

The system can be used for performing regular screening for eye conditions or diseases. The first symptoms of the condition and/or disease can be accurately detected. Glaucoma can for instance be detected in an early stage (while no symptoms have occurred yet). In this way, it can be avoided that this disease is only detected if a patient's vision has already deteriorated. The progress of the disease can in this way be detected and halted in an early phase. This intervention can reduce the risk of irreversible damage to the eyes of the patient.

Optionally, the deep learning neural network is configured to have pixels of the retina image as input, and providing a score indicative of a disease or condition (e.g. glaucoma) as an output.

Optionally, the deep learning neural network is configured to have pixels of the retina image as input, and providing a score indicative of a disease risk (e.g. risk to get a stroke) as an output.

Advantageously, the system enables ophthalmologists to make earlier and equally better analysis and see how a disease/condition progresses.

Optionally, the computer program product can retrieve information from connected scanning devices usable for detecting retinal abnormalities (e.g. used by ophthalmologists).

According to an aspect, the invention provides for a computer-implemented method of classifying images of retina of eyes of subjects, the method comprising operating one or more hardware processors to: receiving an image of a retina captured by means of an imaging unit; segmenting the captured image of the retina in order to obtain at least one segmented image; and providing the at least one segmented image as input to one or more trained machine learning models configured to classify the eye.

The deep neural network can be trained to detect features in the image of the retina which would indicate a disease or condition, even at an early phase. The system can be used for identifying general systemic diseases.

The machine learning model can provide a heat map indicating which region(s) of the image provided most information for detecting the condition/disease. For example, when the image of the retina is not segmented, the machine learning models may solely focus on regions of the retina image covering the optical disc. In this way, the model may not take into account the blood vessels, although the blood vessels may also include relevant information. By providing separate segmented images of the retina to one or more machine learning models, a more thorough and/or accurate analysis of the retina image can be obtained. Optionally, an ensemble model is used which employs different machine learning models to come to a single output indicating a classification of the captured image of the retina.

The segmented image may neutralize information substantially overlapping with identified landmarks in the retina image. Various automatic landmark (e.g. optical disc, blood vessel structure, etc.) location determination algorithms can be employed. In some examples, a trained convolutional neural network is used for localization of one or more landmarks in the image of the retina. However, the location determination algorithms may also involve image processing.

Optionally, the retinal image is further preprocessed prior to feeding it to a machine learning model. For example, at least one of a sharpness, contrast, color balance, color parameters, etc. of the image can be adjusted. Various other parameters can be tuned in the preprocessing. For example, a filter may be employed on the image of the retina.

The one or more segments of the image of the retina can be patched with a uniform color. Different colors can be used (e.g. black, blue, gray, red, etc.). In some examples, a random color is employed. However, it is also possible to patch the one or more segments by using a pattern, a texture, or the like. For example, in some examples a noise pattern can be employed. The convolution neural network identify structures or edges present in the image. Having a smooth surface can result in the convolutional neural network to use other parts of the image to perform the prediction.

In addition to providing more accurate classification of the retina images, the invention also enables to provide for improved explainability and better model interpretability. The machine learning model can be partially pulled open by exclusively directing the model to focus on certain predetermined regions in the retina image. As a result, also the model trustworthiness can be enhanced. As the retina image may have redundant information usable for detecting a certain classification (e.g. a condition/disease of the eye such as glaucoma), the trustworthiness of the classification can be enhanced by providing different exclusive regions (cf. segmented) to the machine learning model. Advantageously, it can be better observed how a condition/disease is linked to a particular part of the retina.

According to an aspect, the invention provides for a computing system which is configured to receive or capture an image of a retina of an eye, segmenting the image of the retina, extracting features from only the segmented image of the retina, and identifying characteristics of the retina based on the extracted features, wherein the image of the retina is classified based on the identified characteristics. A machine learning model, such as a (deep) neural network model can be used for the classification.

According to an aspect, the invention provides for a computing system configured to classify images of eyes of subjects, the classification being usable to infer or further analyze a condition of the subjects, the system including one or more hardware computer processors, and one or more storage devices configured to store software instructions configured for execution by the one or more hardware computer processors in order to cause the system to: receiving an image of a retina captured by means of an imaging unit; removing image data within a first segment area of the captured image of the retina in order to obtain a first segmented image, the first segment area overlapping the optic nerve head in the captured image of the retina; and providing the image of the retina with the image data within the first segment area being removed as input to a trained machine learning model configured to classify the image of the retina.

According to an aspect, the invention provides for a use of the system for diagnosing disorders using retinal images.

It will be appreciated that the selected portions of the captured image may be spatial selections of one or more spatial regions or one or more spatial areas of the captured image. However, it is also envisaged that the selected portions of the captured image correspond to selection information within the captured image, for instance some selected colours may be segmented away and/or filtered out (e.g. some colours of the captured image may be segmented/filtered out to obtain a segmented image).

It will be appreciated that the ensemble classification can be seen as a combined classification which is at least partially based on the first and the second classifications. It is also possible that the ensemble/combined classification is based on one or more further classifications obtained by means of different (dedicated) trained machine learning models.

It will be appreciated that the ensemble/combined classification may be calculated on the basis of the first and second classifications (e.g. averaging, means of predictions, weighted averaging, etc.). It is also envisaged that a machine learning model (e.g. random forest) is used for determining the ensemble/combined classification based on the first and second classifications. For example, at least the first and second classifications may be provided as input to the machine learning model in order to obtain the ensemble/combined classification as an output.

Random forests or random decision forests are an ensemble learning method for classification/regression that operate by constructing a multitude of decision trees at training time and outputting the class that is the mode of the classes (classification) or mean/average prediction (regression) of the individual trees. However other kind of machine learning models may also be employed for determining the ensemble/combined classification, for instance a neural network models.

It will be appreciated that any of the aspects, features and options described in view of the system apply equally to the computer-implemented method and the described imaging apparatus and use. It will also be clear that any one or more of the above aspects, features and options can be combined.

### BRIEF DESCRIPTION OF THE DRAWING

The invention will further be elucidated on the basis of exemplary embodiments which are represented in a drawing. The exemplary embodiments are given by way of non-limitative illustration. It is noted that the figures are only schematic representations of embodiments of the invention that are given by way of non-limiting example.

In the drawing:

FIG. 1 shows a captured image of a retina after contrast enhancement;

FIGS. 2A, 2B, and 2C show segmented images based on a captured image of a retina;

FIGS. 3A and 3B shows an unsegmented and a segmented image of a retina, respectively;

FIG. 4 shows a preprocessed captured image of a retina;

FIG. 5 shows a schematic diagram of an embodiment of a method;

FIG. 6 shows a schematic diagram of an embodiment of a method;

FIG. 7 shows a schematic diagram of an embodiment of a method;

FIG. 8 shows a schematic diagram of an embodiment of a method; and

FIG. 9 shows a schematic diagram of an embodiment of a method.

### DETAILED DESCRIPTION

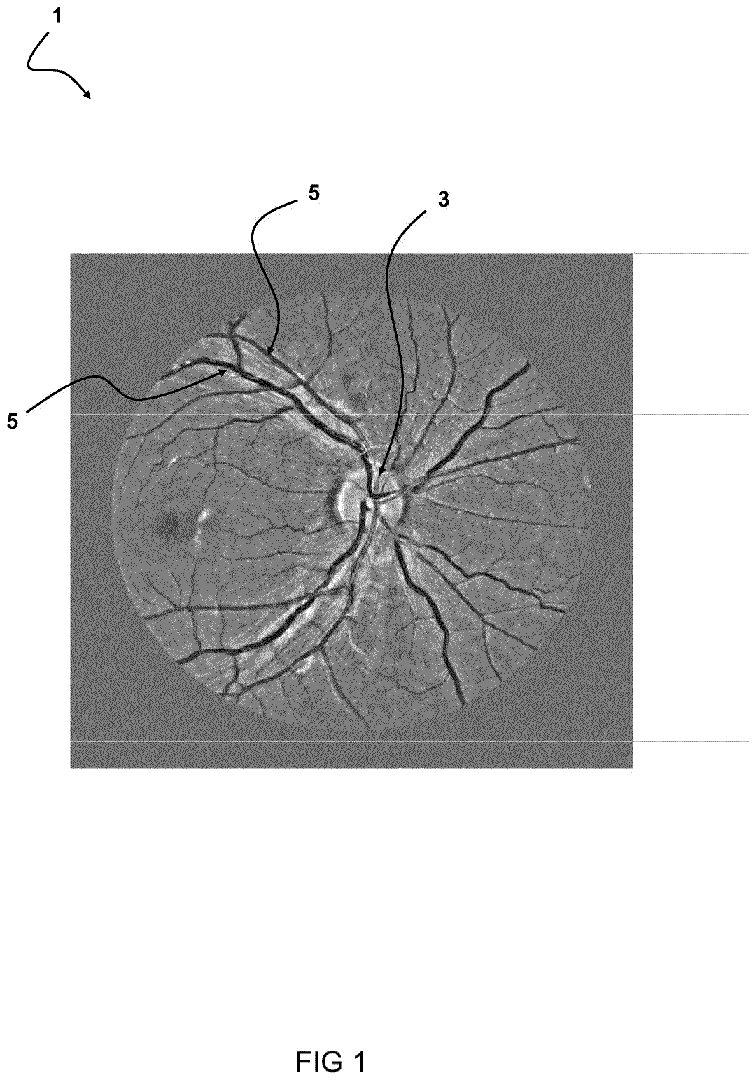

FIG. 1 shows a retina image 1 of an eye (after contrast enhancement). This image can be used by the computing system configured to classify images of retina of eyes of subjects. The system can be configured to receiving the image 1 of the retina captured by means of an imaging unit. Different types of hardware components can be used as the imaging unit. Furthermore, the system can be configured to segmenting the captured image 1 of the retina in order to obtain at least one segmented image. The at least one segmented image can be provided as input to one or more trained machine learning models configured to classify the eye. For example, the classification can be usable to infer or further analyze a condition of subjects.

In the captured retina image 1, an optic nerve head 3 and blood vessels 5 are distinguishable. These features can be seen as landmarks of the retina. Segmentation of the retina image 1 can be performed based on these landmarks, for instance including or excluding these landmarks. It is also envisaged that other landmarks are used for segmentation of the retina image 1. In some examples, some regions of the retina image are segmented. The segmentation can be performed for instance by neutralizing relevant information within segment areas, for instance by applying a uniform pixel color, random pixel values, etc.

A detection algorithm may be employed configured to recognize the segment to be excluded (cf. clipping). The segment can be excluded by neutralizing image data within a segment area. By excluding the segment of the retina image, the remaining information in the image can be used for making the prediction. For instance, the optic nerve may be excluded in a segmented image. The prediction of the disease/condition can then be performed using one or more machine learning models with the segmented image and the unsegmented image. Additionally or alternatively, this can be performed for the blood vessels, and/or other landmarks/features which can be identified in the captured retina image.

Optionally, information covering at least a part of one landmark is neutralized (e.g. deleted) or replaced in the segmented image. The landmark may form a structure or characteristic of the retina which is recognizable by processing of the captured image. The landmark may for instance be an optic nerve, blood vessel structure, etc. The landmark may be covered (for neutralization of data) by using different shapes, for example a circle, rectangular, polygon, ellipse, irregular shape, etc.

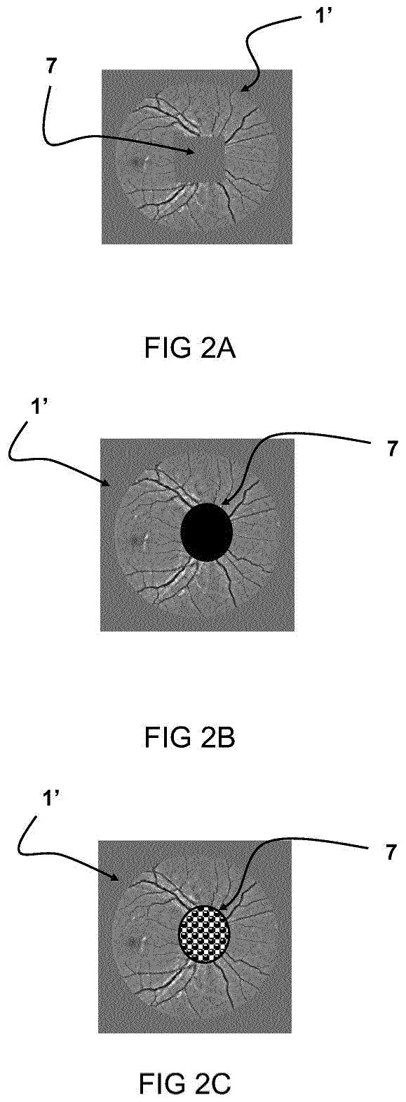

FIGS. 2A, 2B, and 2C show segmented images 1′. In these examples, the captured image 1 as shown in FIG. 1 is segmented. In these examples, the segmented images are segmented to exclude an optic nerve head 3 of the retina in the captured image. However, it is also possible to perform segmentation based on other landmarks identified in the captured image of the retina of the eye. In this example, segmentation in the segmented images is performed by neutralizing (cf. removing) image data within at least one segment area 7 of the captured image 1 of the retina/eye, the at least one segment area 7 covering parts of the eye to be excluded as relevant input for the one or more trained neural networks. This can be performed in various ways. For instance, in FIG. 2A, a segment area 7 substantially covers the optic nerve head with a uniform pixel color value (e.g. grey). The segment area 7 has a rectangular shape in FIG. 2A. The segment area 7 in FIG. 2B has a circular shape. Other shapes are also possible. It is also possible to use other pixel color values for neutralizing relevant image data within the part of the captured image covered by the segment area 7. For example, the pixel color values within the segment area 7 are set to black in FIG. 2B. Instead of employing uniform pixel color distributions, it is also possible to use color gradients, random colors (noise), patterns, or the like. For example, the segment area 7 in FIG. 2C has a regular pattern.

There can be separate information in the optical disc and the vessel structure in the retina image. The separate information in different landmarks/features of the retina can be separately assessed by trained machine learning models, enabling a better prediction. For example, in the captured retina image 1, the vessels may be segmented away (information in the regions covering the vessel structure may be removed by for instance employing a uniform color). The vessel structure can also be fed separately to the machine learning model. It will be appreciated that segmenting a part of the retina image out can be performed in different ways. Additionally or alternatively, the complete unsegmented image of the retina may also be fed to a machine learning model for classification. This allows a distinction to be made between changes to the vessels or optic nerve, which can be useful information for example for further analysis.

For example, a vascular dysfunction can be detected by analyzing the images with focus on the blood vessel structure in the captured image of the retina (optionally preprocessed). The system can also look at the optic nerve of the retina. For example, if there is false positive prediction by mainly taking into account the optic nerve in the image of the retina, a better overall prediction may be obtained by considering other parts of the image of the retina (for example the optic nerve segmented away).

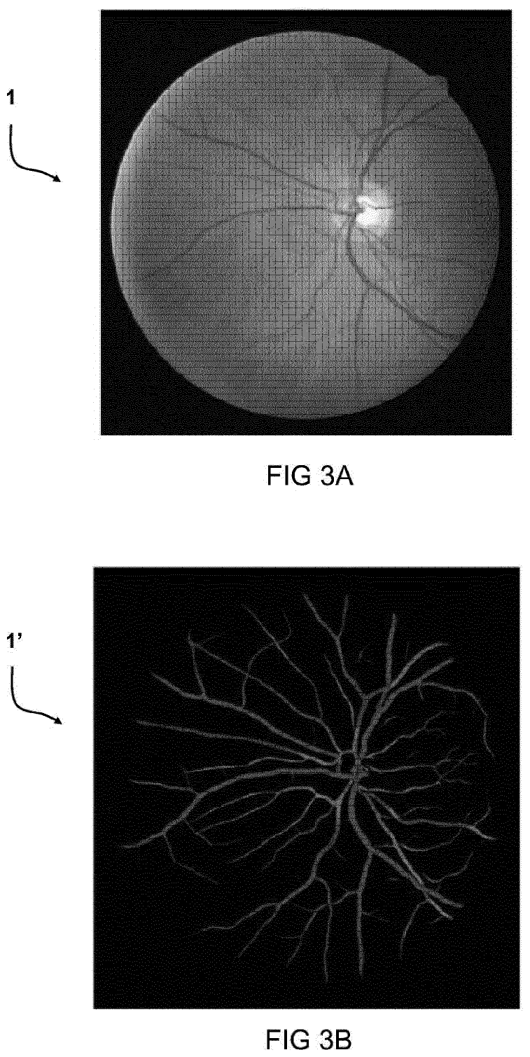

FIG. 3A shows an unsegmented captured image 1 of the retina, and FIG. 3B shows a segmented image 1′ of the retina obtained based on the captured image. In this shown example, the captured image 1 in FIG. 3A is processed for determining the blood vessels of the retina as landmarks. The segmented image 1′ is obtained only including the identified blood vessels with differentiation between arteries (in red) and veins (in blue). It will be appreciated that it is also envisaged that other landmarks are used (e.g. optic nerve head, a sub-group of blood vessels, etc.). Instead of exclusively including data related to the identified blood vessels in the segmented image 1′, it is also possible to exclude this data from the captured image. In such a case, it is possible to perform an analysis which does not take into account data in the blood vessels, forcing the trained neural network to use other regions of the captured image to classify the image of the retina.

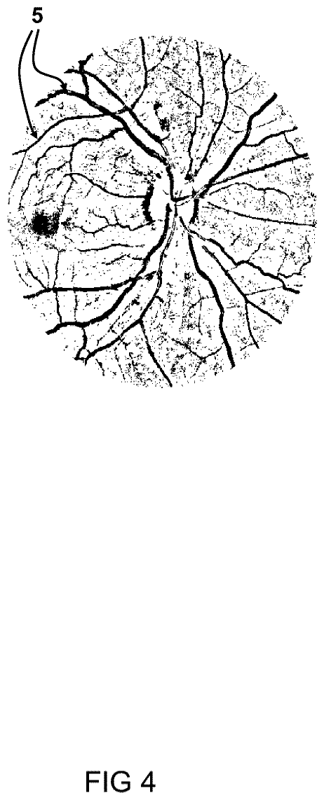

FIG. 4 shows a preprocessed captured image of a retina of an eye. The captured image has been processed such as to more easily identify the blood vessels. The preprocessing may for instance involve image preprocessing in which contrast, sharpness, and other imaging parameters are adjusted for obtaining an enhanced image allowing more easy identification of landmarks features of the retina. It is also possible to use filters or the like. In some examples, additionally or alternatively, a machine learning model is employed for obtaining a preprocessed image.

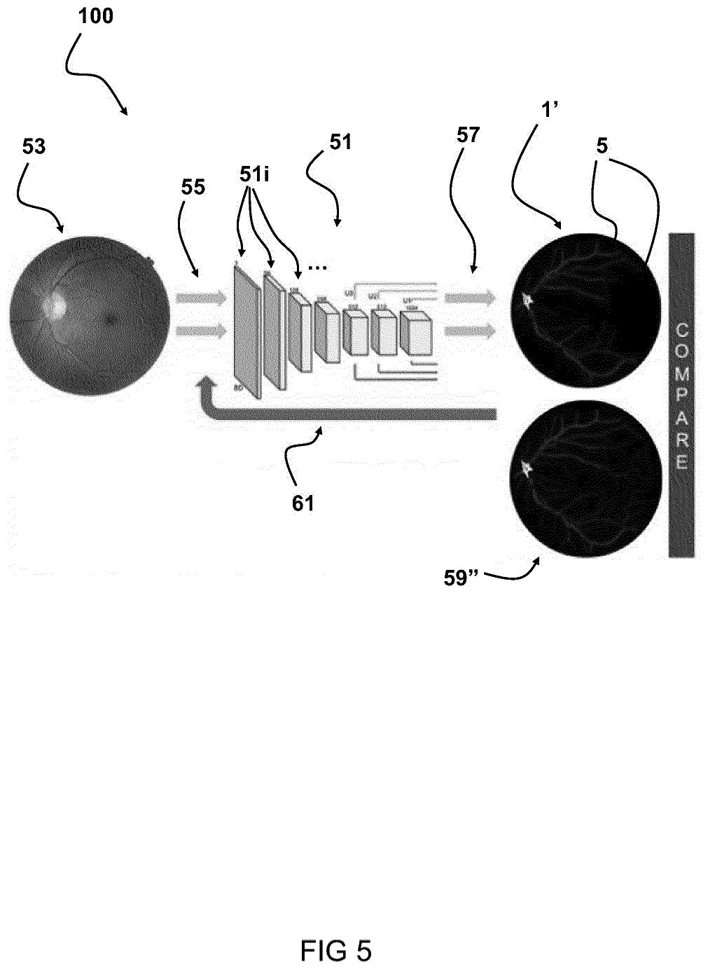

FIG. 5 shows a schematic diagram of an embodiment of a method 50. In the shown exemplary method, a convolutional neural network 51 (CNN) is used for image segmentation. The convolutional neural network 51 includes a plurality of layers 51i. The captured image of the retina 53 is provided as input 55 to the convolutional neural network 51. For example, each pixel value of the captured image may be provided as node input to nodes of a first layer of the CNN 51. The CNN 51 can be configured to provide as output 57 an identification of the blood vessels 5 in the captured image of the retina 53. In this example the CNN is able to distinguish arteries (depicted in red) from veins (depicted in blue). In some examples, the output provides an image with identified blood vessels 5. The output of the CNN 51 can be seen as a prediction. This output can be compared with a ground truth image 59″ for training of the CNN 51. Based on the comparison with the ground truth image 59″, backpropagation 61 may be employed for altering weights of the CNN 51. The ground truth image 59″ may be obtained for instance by means of manual labeling.

The resulting segmented image 1′ can be used as input to a machine learning model for classifying the image of the retina (not shown in this figure). In this example, the segmentation is performed based on the blood vessels, however, it is also envisaged to perform segmentation based on other retina landmarks, for example the optic nerve head, sub-groups of blood vessels, one or more predetermined regions of the retina, etc. By providing segmented images to the machine learning model, data used for classifying the retina image can be more easily controlled. For instance, the machine learning model for classifying the captured image can be forced to look only to the blood vessels of the retina, for example for inferring and/or further analyzing a condition of the subjects.

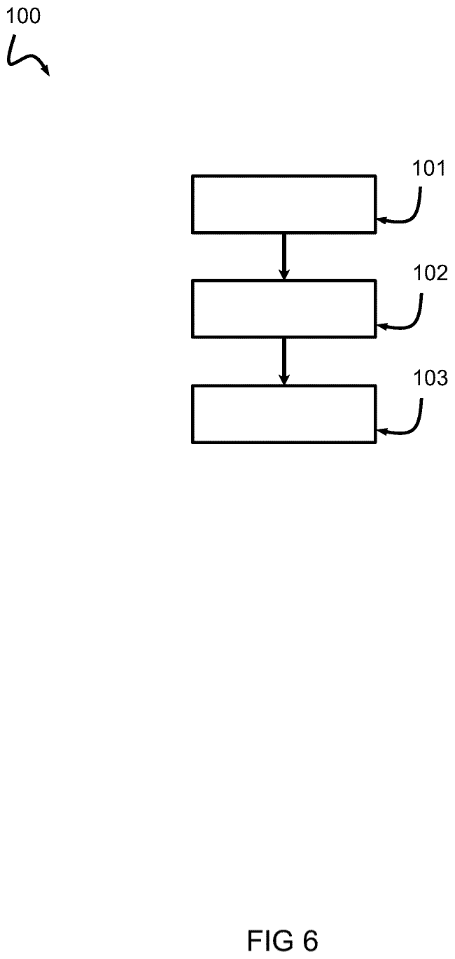

FIG. 6 shows a schematic diagram of an embodiment of a computer-implemented method 100 for classifying images of retina of eyes of subjects. One or more hardware processors are operated for performing the steps of the method 100. In a first step 101, an image of an eye captured by means of an imaging unit is received. In a second step 102, the captured image of the eye is segmented in order to obtain at least one segmented image. In a third step 103, the at least one segmented image is provided as input to one or more trained machine learning models configured to classify the eye.

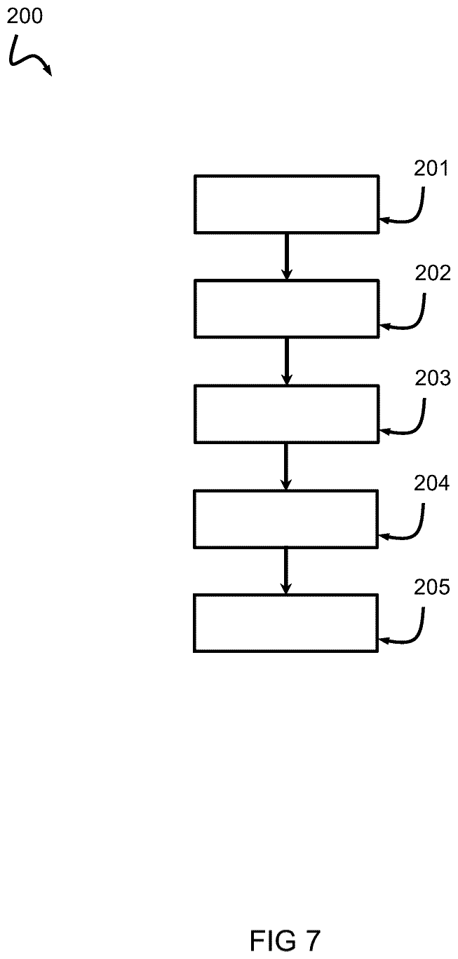

FIG. 7 shows a schematic diagram of an embodiment of a computer-implemented method 200. The method 200 may be carried out on a computing system configured to classify images of retina of eyes of subjects. The system may include one or more hardware computer processors, and one or more storage devices configured to store software instructions configured for execution by the one or more hardware computer processors in order to cause the system to perform method steps according to the invention. In a first step 201, an initial image of a retina captured by means of an imaging unit is received. In a second step 202, the initial image of the retina is processed in order to obtain at least a first segmented image and a second segmented image different from the first segmented image, wherein the first segmented image only includes a first selected portion of the captured image of the retina by employing a first selection rule, and wherein the second segmented image only includes a second selected portion of the captured image of the retina by employing a second selection rule, the first and second selection rules being different with respect to each other. In a third step 203, at least a first trained machine learning model and a second trained machine learning model is provided, each configured to output an image classification based on an input image, the first and second machine learning models being different with respect to each other, and wherein the first machine learning model is trained using segmented images with only selected portions obtained by employing the first selection rule, and wherein the second machine learning model is trained using segmented images with only selected portions obtained by employing the second selection rule. In a fourth step 204, the first segmented image with the first selected portion is provided to the first machine learning model as input image in order to obtain a first classification, and the second segmented image with the second selection portion is provided as input image to the second machine learning model as input image in order to obtain a second classification. In a fifth step 205, an ensemble classification is determined based on at least the first classification and the second classification.

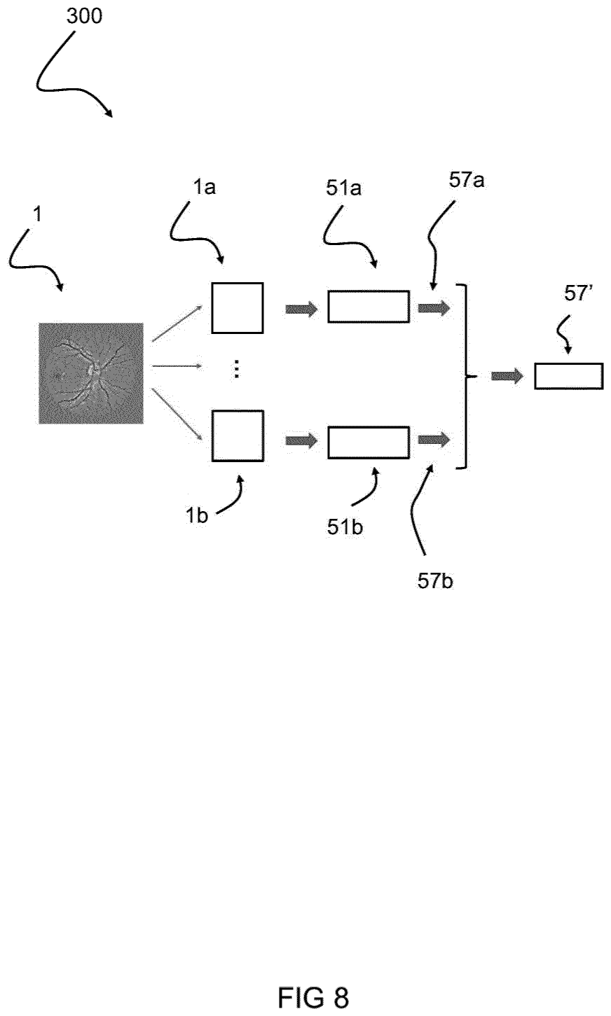

FIG. 8 shows a schematic diagram of an embodiment of a method 300. A captured initial retina image 1 of an eye (optionally preprocessed) is first processed to obtain at least two segmented images. At least first segmented image 1a and a second segmented image 1b different from the first segmented image. The segmented images 1a, 1b may have the same or different resolution with respect to the initial captured image. The first segmented image 1a only includes a first selected portion of the captured image of the retina by employing a first selection rule. In this way, some parts of the initially captured image are excluded or removed in the first segmented image 1a. Similarly, the second segmented image only includes a second selected portion of the captured image of the retina by employing a second selection rule different with respect to other selection rules employed (in this example, different with respect to at least the first selection rule). Furthermore, at least a first trained machine learning model 51a and a second trained machine learning model 51b each configured to output an image classification based on an input image are provided. The first and second machine learning models 51a, 51b are different with respect to each other and separately trained. The first machine learning model 51a is trained using images on which the first selection rule is applied, and the second machine learning model 51b is trained using images on which the second selection rule is applied. Hence, these machine learning models are particularly trained to take into account information in regions remaining in the segmented images by employing the respective selection rule. The first segmented image 1a is provided as input to the first machine learning model 51a, and the second segmented image 1b is provided as input to the second machine learning model 51b. This may be performed in parallel as shown in the figure. As a result, a first classification 57a and a second classification 57b is obtained, respectively. An ensemble classification 57′ is determined based on at least the first classification 57a and the second classification 57b.

It will be appreciated that a larger number of segmented images may be generated following unique selection rules and dedicated machine learning models. The ensemble classification may be based on the larger number of classifications obtained in this way.

Each machine learning model being employed may be trained to predict the same subject property or condition (e.g. a disease, age, health, etc.). The different machine learning model predictions and their confidence levels can be combined to come to a final prediction. Furthermore, this may give insight of where the changes to the retina have happened. Advantageously, the method enables a more accurate prediction/classification. Additionally, the explainability can be improved and more interpretable overall model is obtained. The machine learning method can be made more trustworthy.

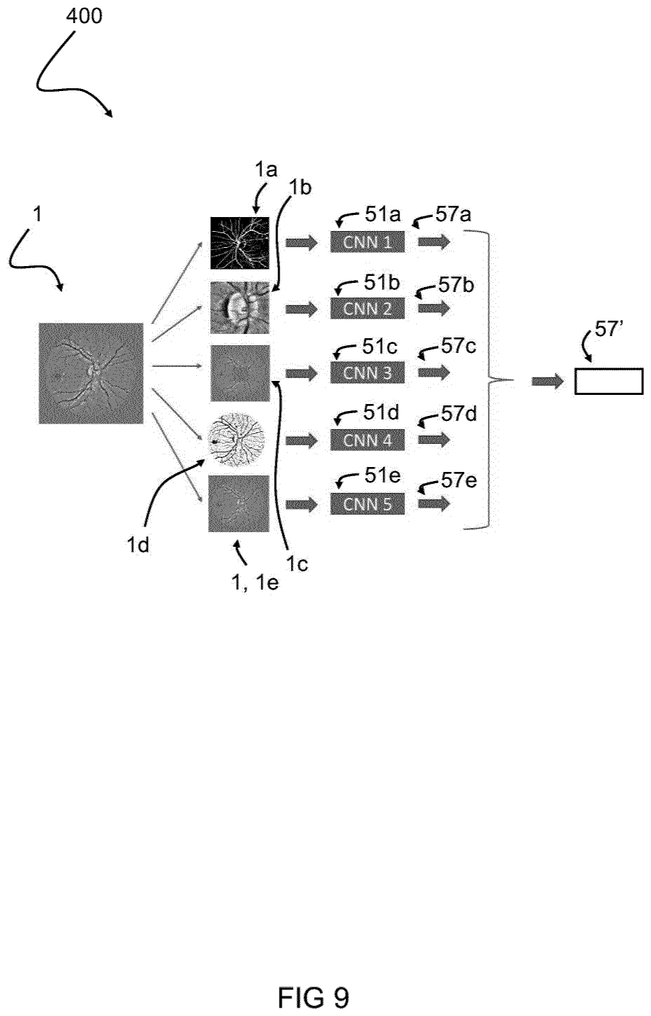

FIG. 9 shows a schematic diagram of an embodiment of a method 400. In this example, the captured initial retina image 1 is processed to obtain four different segmented images 1a, 1b, 1c, 1d. Each of the segmented images is obtained by applying a predefined (unique) selection rule. Similarly as shown in the exemplary embodiment of FIG. 8, each segmented image 1a, 1b, 1c, 1d is provided as input to a different trained machine learning models 51a, 51b, 51c, 51d. These machine learning models 51a, 51b, 51c, 51d are trained by means of training images which are correspondingly generated by similarly applying predefined (unique) selection rules (same selection rule as the respective segmented image 1a-1d provided as input to the machine learning model 51a-51d). Additionally, in this example, also the captured initial retina image 1, 1e is provided to a separate machine learning model 51e. The machine learning model 51e is trained using unsegmented training images of the retina (similar to the captured initial retina image 1). The plurality of machine learning models 51a, 51b, 51c, 51d, 51e may generate respective classifications 57a, 57b, 57c, 57d, 57e as output. An ensemble classification 57′ is determined based on the plurality of classifications 57a, 57b, 57c, 57d, 57e.

In this example, the machine learning models are convolutional neural networks (CNN). It will be appreciated that other types of machine learning models may also be used.

The invention provides for an improved explainability in the decision in condition classification by the deep learning model using fundus images as input. Instead of using a technique in which parts of the test images are perturbed (standard occlusion technique), and the effect on performance recorded, different segmented images are used with obtained by means of different selection rules, each segmented image provided to differently trained machine learning models. One major downside of occlusion testing is the violation of having a similar distribution in train and test sets. When training on a complete image, and evaluating on a perturbed image, it is impossible to assess whether the change in prediction is due to the perturbation or because the omitted information was truly (un)informative.

The invention exploits the importance of other regions next to the regions typically known to have the most relevant data (e.g. ONH). The regions beyond the ONH can also be used and taken into account. An objective explainability in deep learning applications for classification of retina images (e.g. for glaucoma detection) can be provided.

It will be appreciated that the method may include computer implemented steps. All above mentioned steps can be computer implemented steps. Embodiments may comprise computer apparatus, wherein processes performed in computer apparatus. The invention also extends to computer programs, particularly computer programs on or in a carrier, adapted for putting the invention into practice. The program may be in the form of source or object code or in any other form suitable for use in the implementation of the processes according to the invention. The carrier may be any entity or device capable of carrying the program. For example, the carrier may comprise a storage medium, such as a ROM, for example a semiconductor ROM or hard disk. Further, the carrier may be a transmissible carrier such as an electrical or optical signal which may be conveyed via electrical or optical cable or by radio or other means, e.g. via the internet or cloud.

Some embodiments may be implemented, for example, using a machine or tangible computer-readable medium or article which may store an instruction or a set of instructions that, if executed by a machine, may cause the machine to perform a method and/or operations in accordance with the embodiments.

Various embodiments may be implemented using hardware elements, software elements, or a combination of both. Examples of hardware elements may include processors, microprocessors, circuits, application specific integrated circuits (ASIC), programmable logic devices (PLD), digital signal processors (DSP), field programmable gate array (FPGA), logic gates, registers, semiconductor device, microchips, chip sets, et cetera. Examples of software may include software components, programs, applications, computer programs, application programs, system programs, machine programs, operating system software, mobile apps, middleware, firmware, software modules, routines, subroutines, functions, computer implemented methods, procedures, software interfaces, application program interfaces (API), methods, instruction sets, computing code, computer code, et cetera.

Herein, the invention is described with reference to specific examples of embodiments of the invention. It will, however, be evident that various modifications, variations, alternatives and changes may be made therein, without departing from the essence of the invention. For the purpose of clarity and a concise description features are described herein as part of the same or separate embodiments, however, alternative embodiments having combinations of all or some of the features described in these separate embodiments are also envisaged and understood to fall within the framework of the invention as outlined by the claims. The specifications, figures and examples are, accordingly, to be regarded in an illustrative sense rather than in a restrictive sense. The invention is intended to embrace all alternatives, modifications and variations which fall within the spirit and scope of the appended claims. Further, many of the elements that are described are functional entities that may be implemented as discrete or distributed components or in conjunction with other components, in any suitable combination and location.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. A computing system configured to classify images of retina of eyes of subjects, the system including one or more hardware computer processors, and one or more storage devices configured to store software instructions configured for execution by the one or more hardware computer processors in order to cause the system to:
receiving an initial image of a retina captured by means of an imaging unit;
processing the initial image of the retina in order to obtain at least a first segmented image and a second segmented image different from the first segmented image, wherein the first segmented image only includes a first selected portion of the captured image of the retina by employing a first selection rule, and wherein the second segmented image only includes a second selected portion of the captured image of the retina by employing a second selection rule, the first and second selection rules being different with respect to each other;
providing at least a first trained machine learning model and a second trained machine learning model each configured to output an image classification based on an input image, the first and second machine learning models being different with respect to each other, and wherein the first machine learning model is trained using segmented images with only selected portions obtained by employing the first selection rule, and wherein the second machine learning model is trained using segmented images with only selected portions obtained by employing the second selection rule;
providing the first segmented image with the first selected portion to the first machine learning model as input image in order to obtain a first classification, and providing the second segmented image with the second selection portion as input image to the second machine learning model as input image in order to obtain a second classification; and
determining an ensemble classification based on at least the first classification and the second classification.

2. System according to claim 1, wherein the first and second selection rules are configured such that the first and second selected portions of the captured image have no overlap.

3. System according to claim 1 or 2, wherein the second selection rule is configured to provide at least partially inverted selection with respect to the first selection rule.

4. System according to claim 1, 2 or 3, wherein the first selection rule is based on identification of an optic nerve head of the eye, and wherein the second selection rule is based on an identification of blood vessels of the eye.

5. System according to any one of the preceding claims, wherein the first selection rule is configured to selectively exclude a region covering an identified optic nerve head of the eye in the captured initial image, wherein the second selection rule is configured to selectively include a region covering the identified optic nerve head of the eye in the captured initial image.

6. System according to any one of the preceding claims 1-4, wherein the first selection rule is configured to selectively exclude a region covering identified blood vessels of the eye in the captured initial image, wherein the second selection rule is configured to selectively include a region covering the identified blood vessels of the eye in the captured initial image.

7. System according to any one of the preceding claims, wherein the initial image of the retina is processed in order to obtain one or more further segmented images, wherein each of the one or more further segmented images only includes a further selected portion of the captured image of the retina by employing a further selection rule, the further selection rule being different with respect to other selection rules being employed; wherein one or more further trained machine learning models are provided configured to output an image classification based on an input image, each of the one or more further trained machine learning models being different with respect to other trained machine learning models being provided, wherein each of the one or more further machine learning models is trained using further segmented images with only further selected portions obtained by employing the further selection rule; wherein the one or more further segmented images are provided to the respective further machine learning models as input image in order to obtain one or more respective further classifications; wherein the ensemble classification is further based on the one or more further classifications.

8. System according to any one of the preceding claims, wherein at least four different trained machine learning models are employed each configured to receive a respective different segmented image obtained by employing different respective selection rules based on one or more eye features in the captured image.

9. System according to claim 7 or 8, wherein at least two selection rules are employed based on at least two of:
an exclusion of region covering an identified optic disc in the captured initial image;
an exclusion of region covering identified blood vessels in the captured initial image;
an inclusion of region only covering an identified optic disc in the captured initial image; and
an inclusion of region only covering identified blood vessels in the captured initial image.

10. System according to any one of the preceding claims, wherein segmentation in segmented images is performed by removing image data within at least one segment area of the captured image of the eye, the at least one segment area covering parts of the eye to be excluded.

11. System according to any one of the preceding claims, wherein removing of image data is performed by applying a patch over the at least one segment area.

12. System according to claim 10 or 11, wherein the image data within the segment area is removed by setting at least one of a normal distribution, a random distribution, or a uniform distribution of pixel values within the at least one segment area of the captured image.

13. System according to any one of the preceding claims, wherein the unsegmented captured image is further provided as input to the one or more trained machine learning models for classifying the eye.

14. The system according to any one of the preceding claims, wherein the classification is usable to infer or further analyze a condition of the subjects.

15. A computer-implemented method of classifying images of retina of eyes of subjects, the method comprising operating one or more hardware processors to:
receiving an initial image of a retina captured by means of an imaging unit;
processing the initial image of the retina in order to obtain at least a first segmented image and a second segmented image different from the first segmented image, wherein the first segmented image only includes a first selected portion of the captured image of the retina by employing a first selection rule, and wherein the second segmented image only includes a second selected portion of the captured image of the retina by employing a second selection rule, the first and second selection rules being different with respect to each other;
providing at least a first trained machine learning model and a second trained machine learning model each configured to output an image classification based on an input image, the first and second machine learning models being different with respect to each other, and wherein the first machine learning model is trained using segmented images with only selected portions obtained by employing the first selection rule, and wherein the second machine learning model is trained using segmented images with only selected portions obtained by employing the second selection rule;
providing the first segmented image with the first selected portion to the first machine learning model as input image in order to obtain a first classification, and providing the second segmented image with the second selection portion as input image to the second machine learning model as input image in order to obtain a second classification; and
determining an ensemble classification based on at least the first classification and the second classification.
